(19) United States
(12) Patent Application Publication
(10) Pub. No.: US 2025/0222030 A1
(43) Pub. Date: Jul. 10, 2025

(54) NK cells for Cell Therapy

(71) Applicant: Oslo Universitetssykehus HF, Oslo (NO)

(72) Inventors: Sebastién Wälchli, Oslo (NO); Clara H Klee, Oslo (NO)

(73) Assignee: Oslo Universitetssykehus HF, Oslo (NO)

(21) Appl. No.: 19/006,826

(22) Filed: Dec. 31, 2024

(30) Foreign Application Priority Data
Jan. 4, 2024 (NO) 20240016

(51) Int. Cl. A61K 35/17; A61K 40/15; A61K 40/32; A61K 40/42; A61P 35/00; C07K 14/725; C07K 16/30; C12N 5/0783

(52) U.S. Cl. CPC A61K 35/17; A61K 40/15; A61K 40/32; A61K 40/4271; A61P 35/00; C07K 14/7051; C07K 16/3053; C12N 5/0646; A61K 2239/13; A61K 2239/27; C07K 2317/622; C12N 2510/00

(57) ABSTRACT

The present disclosure provides NK cells useful for therapy. A Natural Killer (NK)-cell includes nucleic acids for expressing an αβ T-cell Receptor (TCR), CD3ζ, CD3γ, CD3δ and CD3ε in its cell membrane, and a nucleic acid for expressing and secreting a bispecific protein, wherein the bispecific protein includes a first Fv for binding to a first target epitope and a second Fv for binding to a second target epitope, wherein the first Fv specifically binds, under physiological conditions, to an epitope located on the extracellular part of a CD3-chain, and wherein the second Fv specifically binds, under physiological conditions, to an epitope located on target cells.

### CROSS-REFERENCE TO RELATED APPLICATIONS

This application claims priority to Norwegian Application NO20240016 filed on Jan. 4, 2024, which is incorporated herein by reference in its entirety.

### SEQUENCE LISTING

The Instant Application contains a Sequence Listing which has been submitted electronically in XML format and is hereby incorporated by reference in its entirety. Said XML copy, created on Dec. 19, 2024, is named “NK-TCR with BiTE Sequence listing.xml” and is 14,991 bytes in size. The Sequence Listing does not go beyond the disclosure in the application as filed.

### FIELD OF THE DISCLOSURE

The present disclosure broadly relates to immunotherapy, in particular to cell therapy involving NK cells.

### BACKGROUND

T cells have been used in cell therapy. Naturally occurring T cells express a T cell receptor (TCR). Such cells can be engineered to express other receptors. One example is chimeric antigen receptors (CARs) comprising a targeting unit from antibodies, usually a single-chain Fv (scFv). Several drugs have been approved in USA based on T cells expressing CARs, for example Yescarta®.

In contrast to most conventional antibodies and conventional CARs, TCRs can recognize and bind intracellular target epitopes because proteins are degraded in the cells and peptides from such degradation are presented on the major histocompatibility complex (MHC) on the cell surface.

Considering that all the proteins expressed by a given cell may be degraded into peptides presented on an MHC, TCRs can potentially recognize the whole proteome. This represents a striking numerical advantage over conventional CARs in terms of possible targets. In addition, TCRs can be specifically directed against a mutant variant of a protein while sparing the wild type form. Accordingly, the TCR can distinguish cancer cells expressing the mutated protein from healthy cells expressing the non-mutated protein.

On the other hand, TCRs are complicated molecules to manipulate: they are heterodimers composed of an α- and a β-chain, they do not signal by themselves but require a battery of signalling proteins associated to recruit all the components to create an immune synapse. In addition, their localization in the plasma membrane is believed to depend on co-expression of CD3-chains.

Consequently, TCR-based cell therapy has primarily relied on T cells. However, recombinant expression of TCRs may compete with the endogenous TCR for the “use” of the signalling proteins. Another issue with the introduction of a second TCR into T-cell, is the risk associated with the possibility of forming mixed dimers, thus reducing the number of correct or therapeutic TCR molecules expressed in the cell. In addition, mispairing might generate novel TCRs which could potentially be toxic. Although such “mispairing” of TCRs has yet to be documented in a clinical setting, an important number of innovations has been developed in order to prevent this.

Natural Killer (NK) cells were discovered almost 50 years ago and identified by their ability to recognize and kill tumor cells without the requirement of prior antigen exposure. Since their discovery, NK cells have grown as promising agents for cell-based cancer immunotherapies. However, naturally occurring NK cells do not express TCRs.

### SUMMARY

The present disclosure provides NK cells expressing a TCR-complex and secreting a bispecific protein (see FIG. 1). Such cells, as described herein, will provide cytotoxic activity against target cells, (see FIG. 5) while also being able to recruit endogenous T cells in the patient to provide additional cytotoxic activity to same target cells. Furthermore, they can provide an autocrine stimulation (see FIG. 6), i.e., that the bispecific protein improves the cytotoxic activity of the NK-cell. In particular, the TCRs may recognize intracellular target proteins when their degradation products are presented by MHC on the target cells while the bispecific proteins may recognize surface proteins on the target cells. As demonstrated herein, such in situ production of bispecific proteins such as BiTEs will allow an improved immune response to the target cells. This can be especially beneficial in solid tumors. If (i), the TCR and the BiTE recognize different epitopes of the same target protein, the target cells will be less likely to avoid the immune response based on loss of human leukocyte antigen complex (HLA) or other loss of surface expression. If (ii), the TCR and the BiTE recognize epitopes of different target proteins from the same target cells, these target cells can be extra sensitive due to a “crossfire mechanism”, e.g., by targeting key proteins in two different pathways. Furthermore, the latter approach (ii), may be efficient in situations where there is heterogeneity in target expression. If one target is lost from the target cell, the second target may still be present. Antigen loss is known in many cancers, and it may be relevant between metastases as well.

The present disclosure provides a Natural Killer cell comprising nucleic acids for expressing an αβ T-cell Receptor, CD3ζ, CD3γ, CD3δ and CD3ε in its cell membrane, and nucleic acid for expressing and secreting a bispecific protein, wherein the bispecific protein comprises a first Fv for binding to a first target epitope and a second Fv for binding to a second target epitope, wherein the first Fv specifically binds, under physiological conditions, to an epitope located on the extracellular part of a CD3-chain, and wherein the second Fv specifically binds, under physiological conditions, to an epitope located on target cells.

In one aspect, the TCR and the bispecific protein recognize different epitopes of the same target protein from the same target cells.

In one aspect, the TCR and the bispecific protein recognize epitopes of different target proteins from the same target cells.

In one aspect, the TCR specifically binds, under physiological conditions, to a peptide from Melanoma-associated antigen 4 presented on an HLA, and the second Fv specifically binds, under physiological conditions, to an extracellular target epitope located on a human cancer cell selected from a group consisting of non-small cell lung cancer, ovarian cancer, melanoma cancer, synovial sarcoma and gastroesophageal cancers.

In one aspect, the TCR specifically binds, under physiological conditions, to a peptide from Kita-Kyushu Lung Cancer Antigen-1 presented on an HLA, and the second Fv specifically binds, under physiological conditions, to an extracellular target epitope located on a human cancer cell selected from a group consisting of breast cancer, gastric cancer, lung cancer, pancreatic cancer and cervix cancer.

In one aspect, the TCR specifically binds, under physiological conditions, to a peptide from PRAME presented on an HLA, and the second Fv specifically binds, under physiological conditions, to an extracellular target epitope located on a human melanoma cell.

In one aspect, the first Fv specifically binds, under physiological conditions, to an extracellular epitope located on a human CD3ε protein.

In one aspect, the second Fv specifically binds, under physiological conditions, to an extracellular epitope located on human cancer cells of solid tumor origin. One example of a BiTE comprising such Fv is provided by SEQ ID NO: 3 (the TP3 BiTE).

In one aspect, the first and/or second Fv is a single-chain Fv.

In one aspect, the NK cell is a human cell line or human primary cell.

The present disclosure also provides a cell population comprising the cells disclosed above.

The present disclosure also provides an aqueous pharmaceutical composition for intravenous or intratumoral administration comprising the cells disclosed above or the cell population disclosed above, wherein the composition is sterile and/or isotonic.

The present disclosure also provides a method of treatment of solid tumors comprising the step of administering the pharmaceutical composition disclosed above to a human patient in need thereof.

### BRIEF DESCRIPTION OF THE FIGURES

FIG. 1 shows that a classical BiTE can be added exogenously to the T cells which will guide them to the target. A T-BiTE is a T cell that expresses a transgene encoding for a BiTE molecule which will be released and stimulate the expressing T cell and the surrounding ones. NK-TCR-BiTE are NK cells that will also release BiTEs; these BiTEs will guide NK cells only if they express a TCR-complex at their surface and any by-standing T cell present at the tumour site.

FIG. 2 shows, in a simplified manner, a TCR-complex in a NK-cell membrane. The TCR-complex comprises an α-chain with three CDRs and a β-chain with three CDRs together forming an αβTCR. The TCR-complex also comprises two CD3ζ-chains, two CD3ε-chains, one CD3γ-chain and one CD3δ-chain. A secreted BiTE comprising two different scFv's connected by a linker is also shown.

FIGS. 3a-d show, in a simplified manner, a conventional antibody, FIG. 3a, comprising two heavy chains and two light chains. Each light chain is connected to a heavy chain by a cysteine bridge, and the heavy chains are connected to each other by two cysteine bridges. The Variable domains forms two identical Fv's. The N-terminal of the chains is represented by an N. The C-terminal of the chains is represented by a C. Each Fv comprises a VL and a VH with three CDRs (see FIG. 3b). The VL and VH can be connected from C-terminal to the N-terminal by a peptide linker represented by a diagonal line, to form a scFv. The two orientations VL-linker-VH and VH-linker-VL are visualized in FIGS. 3c and 3d.

FIG. 4 shows, in a simplified manner, a BiTE comprising two scFv's connected with a linker. Each of the VLs and the VHs comprise three CDRs which are visualized by three gray sections. The N-terminal of the chain is represented by an N. The C-terminal of the chain is represented by a C.

FIG. 5 shows how a NK-TCR-BiTE efficiently acquire BiTE specificity and are efficient at killing BiTE target. Here NK-92-TCR were tested in a BLI killing assay against BL41 cells and showed limited reactivity (filled circle) against this cell line. However, when they express BiTEs, they recognize and kill B cell in a CD19-(square) or CD22-(circle) dependent manner. It demonstrates that BiTEs are produced in NK cells. It further demonstrates that BiTEs can activate NK-TCR cells to kill a target, since the NK-TCR not producing BiTE cannot kill.

FIG. 6 shows a sorted population tested for CD19 killing. The TCR expressed in these NK-TCR-cells is Radium-1 (Rad-1). This TCR is specific for a frameshift mutation in the transforming growth factor β receptor II (TGFβRII) present in the majority of MSI+ colon cancers. We selected cells sensitive to Rad-1 (e.g. HCT-116 cells) and modified them to express CD19. The killing efficiency of NK-TCR is increased against HCT-116-CD19 only when NK-TCR express CD19-BiTE i.e. a BiTE specific for CD19, suggesting that the BiTE presence increases killing. It demonstrates an autocrine stimulation of NK-TCR-cells by the secreted BiTE, providing an improved cytotoxic effect against target cells.

FIG. 7 shows the production of BiTEs from a transgene. A polycistronic gene will be transcribed as a long mRNA, which will be translated into two proteins separated by a 2A-ribosome skipping sequence. Here, the first protein will be the BiTE composed of two scFv linked by a linker sequence and a His-tag fused at the C-terminal end of the second scFv. Due to the presence of a signal peptide at the N-terminal end, the BiTE will be exported to the exocytosis machinery and release in the cell medium. After the His-tag coding sequence, the 2A-ribosome skipping sequence will lead to the release of the BiTE and the translation of the second gene, here GFP. This fluorescent protein will stay in the cytosol and will be used to detect BiTE-expressing cells.

FIG. 8 shows expression of BiTE in NK cells by GFP detection. NK-TCR cells transduced or not with CD19 BiTE were run into a flowcytometer and fluorescence was detected using laser excitement at 488 nm and detected at 510 nm.

FIG. 9 shows a Western blot of BiTEs as described in Example 2. A nickel slurry was used to pull down His-tagged BiTE from the supernatant and each fractions were separated in an SDS-PAGE and transfer to a nitrocellulose membrane for Western blot detection using an anti-His antibody. As a control, a validated and purified BiTE solution was used. One mL of a NK cell suspension of ca 1 million cells/mL was isolated and run into a Ni-NTA column to isolate His-tagged proteins. The column was washed, and the first supernatant was spared. The beads were resuspended in SDS-PAGE loading buffer. The indicated fractions were separated on an SDS-PAGE and transferred to a nitrocellulose membrane for Western blot identification of the BiTE proteins using anti-His antibodies. As shown only the NK-TCR-BiTE cells released BiTE into the medium.

FIG. 10 shows a killing-assay involving coculture of the indicated NK-92 and OSA cells. The NK-92 cells express CD3, CD3+TCR or CD3+TCR+BiTE, only the last one is able to kill. Accordingly, this experiment demonstrates that NK cells expressing a TCR and excreting a BiTE targeting two different solid tumor targets, ALPL-1 and HER2 respectively, provide specific cytotoxic activity.

### DETAILED DESCRIPTION

Natural killer cells, also known as NK cells, are a type of cytotoxic lymphocyte within the innate immune system. NK cells generally provide rapid responses to virus-infected cell and other intracellular pathogens compared to T cells in the vertebrate adaptive immune response. Accordingly, NK cells may provide a first-line defense against tumor formation.

While T cells detect the antigen presented on major histocompatibility complex (MHC) on infected cell surfaces, NK cells can recognize and kill target cells in the absence of antibodies and MHC, allowing a faster immune reaction.

NK cells were named “natural killers” because they do not require activation to kill cells that are missing “self” markers of MHC class I. This is important because harmful cells that are missing MHC I markers cannot easily be detected and destroyed by T cells.

NK cells differentiate from CD127+ common innate lymphoid progenitors, which are downstream of the common lymphoid progenitor from which B and T lymphocytes are also derived. NK cells are known to differentiate and mature in the bone marrow, lymph nodes, spleen, tonsils, and thymus, where they then enter into the circulation.

Naturally occurring NK cells express CD56 in their cell membrane, but they are CD3ε-negative in antibody-assays.

NK cells in the present disclosure differ from natural killer T cells (NKTs) phenotypically, by origin and by respective effector functions. NKT-cell activity promotes NK-cell activity by secreting interferon gamma. In contrast to NKT cells, naturally occurring NK cells do not express TCRs or CD3ε in their cell membrane. However, they usually express the surface markers CD16 and CD57 in humans, NK1.1 or NK1.2 in C57BL/6 mice. The NKp46 is also an NK cell marker.

The term “NK T cells” was first used in mice to define a subset of T cells that expressed the NK-cell-associated marker NK1.1 (CD161). It is now generally accepted that the term “NKT cells” refers to CD1d-restricted T cells, present in mice and humans, some of which co-express a heavily biased, semi-invariant T-cell receptor and NK-cell markers.

In the last decade, several NK-cell based anti-cancer products have undergone clinical trials yielding promising results. However, in order to develop alternative NK-cell products for therapy, novel strategies for increasing safety, efficiency, and specificity are needed.

Introduction of a functional TCR complex to NK cells that can inherently detect and eliminate virally infected and/or tumor cells enhances the efficiency of identification and killing of target cells as well as circumventing potential risk of TCR mispairing. The recombinant technology for expressing TCR-complexes in NK cells by co-expression of TCRs and CD3-chains is thoroughly described in Mensali et al 2019, EBioMedicine. 2019 February:40:106-117. doi: 10.1016/j.ebiom.2019.01.031. As disclosed in WO2018129199A1, recombinant expression of CD3γ, CD3δ and CD3ε together with a TCR is sufficient for expressing TCR-complexes in the cell membrane of NK cells.

The present disclosure primarily concerns human cells as a starting material. Such cells may be isolated from a healthy subject by leukapheresis or other suitable methods. Cells isolated from a healthy subject are primary cells. However, other non-human mammalian cells can also be used.

Culturing of primary NK cells can be challenging, but it is disclosed in Mu et al., BMC Biotechnology, 19, Article number 80 (2019), Klingmann, Cytotherapy, vol. 17, Issue 3, March 2015, p. 245-249, Koepsell et al., Transfusion, vol. 53, issue 2, February 2013, p. 404-410. However, culturing of “immortalized” cells i.e. cell-lines, is generally easier for consistently obtaining larger cell populations. The human cells in the present disclosure can for example be cell-lines like NK-92 or YTS. Such cell lines are commercially available from the American Type Culture Collection (ATCC).

WO2015054299A1 discloses a protocol for culturing as well as media for storage and transport of NK-92 cell line. Said disclosure relates generally to a cell storage medium for transport and/or delivery of cells. For example, it relates to a sterile, isotonic NK-92 cell storage medium for transport and delivery of NK-92 cells, the storage medium comprising: human serum or human serum albumin; an initial density of non-irradiated, substantially non-aggregated NK-92 cells sufficient to provide a therapeutic amount of NK-92 cells at the time of delivery to a treatment facility; and an IL-2 concentration of about 200 IU/mL wherein said medium is maintained within +/−5° C. of a temperature selected for transport, and further wherein said temperature selected for transport is between about 20° C. to about 40° C. in the presence of sufficient oxygen to maintain viability of the cells, such that the NK-92 cells remain viable for administration to a patient up to a period of at least 24 hours after placement into the storage medium.

TCRs are well-known receptors. Their variable domains, either Vα and Vβ or Vγ and Vδ, form a single antigen binding unit based on six complementarity determining regions, CDRs. The CDRs are flanked by framework sequences. The first framework sequence is N-terminal to the CDR1, the second framework sequence is located between CDR1 and CDR2, while the third framework sequence is located between CDR2 and CDR3. Accordingly, both a Vα and a Vβ can be roughly visualized as follows, with the CDRs boxed and the N-terminal indicated as N-:
N-FRAMEWORK1 [symbol] FRAMEWORK2 [symbol] FRAMEWORK3 [symbol] FRAMEWORK4

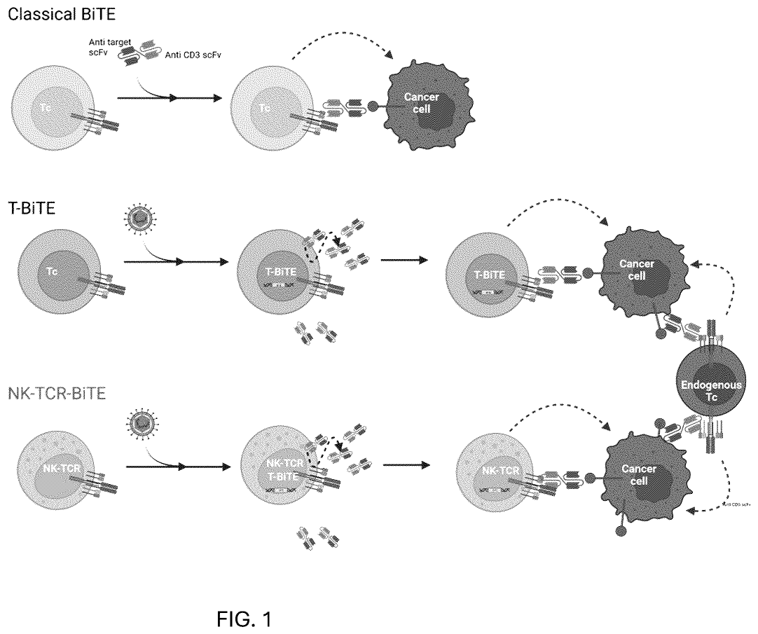

However, the detailed mechanism of action of TCRs is not completely understood. As visualized in FIG. 1, they are believed to form a TCR-complex upon specific binding to its target. Such TCR-complex involves interaction between the TCR and several CD3-chains.

The TCR target epitopes are peptides presented by MHC. TCRs can specifically bind their target epitopes when the cognate peptide is presented by MHC, but they will show little or no binding to MHC when no peptide is presented, or a different peptide is presented.

MHC class I presents peptides comprising 8 to 10 amino acid residues from inside the cell. MHC class II presents peptides comprising 13 to 25 amino acid residues from outside the cell. The human MHC is called HLA (human leukocyte antigen complex).

In one aspect, the NK cells disclosed herein can express TCRs that specifically bind, under physiological conditions to cognate peptides when presented by HLA-A, HLA-B or HLA-B (i.e. MHC class I). These cognate peptides can represent “intracellular target epitopes”.

In one aspect, the NK cells disclosed herein can express TCRs that specifically bind, under physiological conditions to cognate peptides when presented by HLA-DP, HLA-DM, HLA-DQ, HLA-DR (i.e. MHC class II).

Accordingly, in one aspect, the NK-cell disclosed herein expresses a TCR-complex in the cell membrane, wherein the TCR specifically binds, under physiological conditions to a cognate peptide when presented by MHC class I on cancer cells, and wherein the NK-cell secretes a bispecific protein comprising a first targeting unit for specific binding, under physiological conditions, to a target epitope located on the extracellular part of a CD3-chain, and a second targeting unit for specific binding, under physiological conditions, to a target epitope located on cancer cells.

Accordingly, in one aspect, the NK-cell disclosed herein expresses a TCR-complex in the cell membrane, wherein the TCR specifically binds, under physiological conditions to a cognate peptide when presented by MHC class I on cancer cells, and wherein the NK-cell secretes a bispecific protein comprising a first Fv for binding to a first target epitope and a second Fv for binding to a second target epitope, wherein the first Fv specifically binds, under physiological conditions, to an epitope located on the extracellular part of a CD3ε-chain, and wherein the second Fv specifically binds, under physiological conditions, to an epitope located on cancer cells.

Accordingly, in one aspect, the NK-cell disclosed herein expresses a TCR-complex in the cell membrane, wherein the TCR specifically binds, under physiological conditions to a cognate peptide when presented by MHC class I on cancer cells, and wherein the NK-cell secretes a bispecific protein comprising a first Fv for binding to a first target epitope and a second Fv for binding to a second target epitope, wherein the first Fv specifically binds, under physiological conditions, to an epitope located on the extracellular part of a CD3δ-chain, and wherein the second Fv specifically binds, under physiological conditions, to an epitope located on cancer cells.

Accordingly, in one aspect, the NK-cell disclosed herein expresses a TCR-complex in the cell membrane, wherein the TCR specifically binds, under physiological conditions to a cognate peptide when presented by MHC class I on cancer cells, and wherein the NK-cell secretes a bispecific protein comprising a first Fv for binding to a first target epitope and a second Fv for binding to a second target epitope, wherein the first Fv specifically binds, under physiological conditions, to an epitope located on the extracellular part of a CD3γ-chain, and wherein the second Fv specifically binds, under physiological conditions, to an epitope located on cancer cells.

Accordingly, in one aspect, the NK-cell disclosed herein expresses a TCR-complex in the cell membrane, wherein the TCR specifically binds, under physiological conditions an “intracellular target epitope” when the peptide presented by MHC class I on cancer cells, and wherein the NK-cell secretes a bispecific protein comprising a first Fv for binding to a first target epitope and a second Fv for binding to a second target epitope, wherein the first Fv specifically binds, under physiological conditions, to an epitope located on the extracellular part of a CD3ε-chain, and wherein the second Fv specifically binds, under physiological conditions, to an extracellular target epitope located on the same cancer cells.

Accordingly, the modified NK cells disclosed herein can display an MHC-restricted antigen-specific cytotoxicity and they may be useful in therapy of cancer, viral infections, autoimmunity, and graft versus host disease (GvHD).

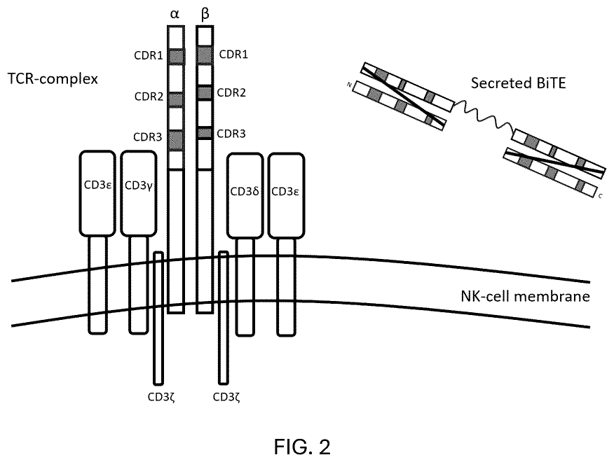

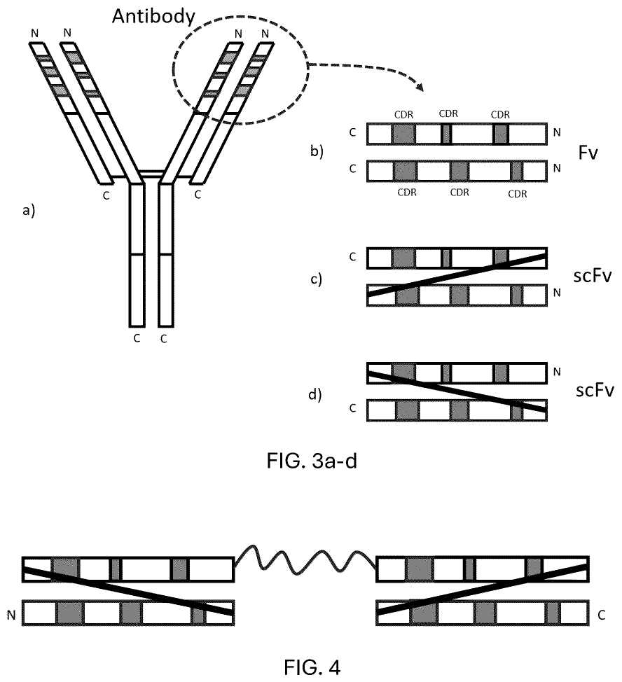

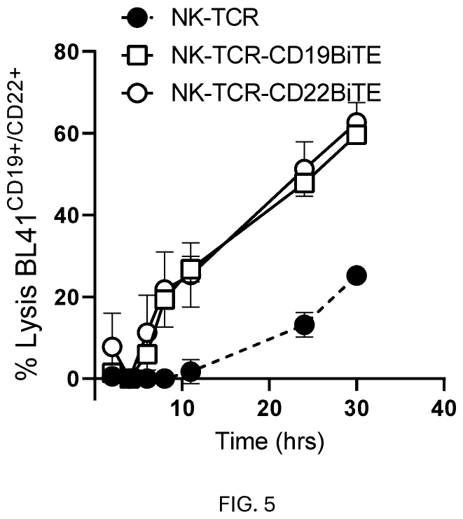

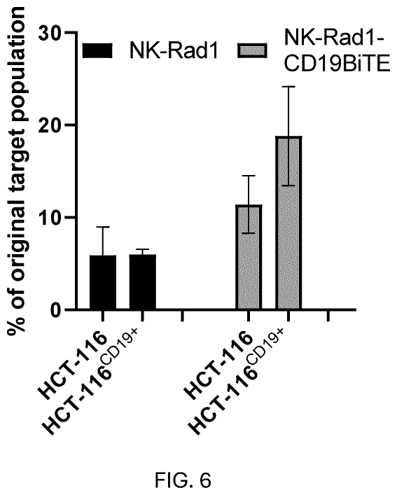

In contrast to TCRs, most naturally occurring antibodies comprise two identical Fv's (see FIG. 2). Because both Fv's are able to bind the same target epitope, such antibodies are monospecific and their binding to the target epitope is bivalent.

An Fv is a protein moiety able to bind a target epitope under physiological conditions. The Fv comprises an antibody light chain variable domain (VL) and an antibody heavy chain variable domain (VH). Such variable domains are well-known for skilled persons.

Provided herein are NK cells expressing and secreting a protein comprising a first targeting unit for binding to a first target epitope and a second targeting unit for binding to a second target epitope. Such proteins are bispecific.

The first and second targeting unit may have any format as long as they provide specific binding, under physiological conditions, to their target epitopes. Such binding units may be obtained by phage display libraries by conventional screening.

They can also be obtained from TCRs. However, more conventionally, such targeting units may be obtained from antibodies of any type including single-domain antibodies.

In particular, provided herein are NK cells expressing and secreting proteins comprising a first Fv for binding to a first target epitope and a second Fv for binding to a second target epitope. Of course, the first and second target epitope are not identical.

In particular, provided herein are NK cells expressing and secreting proteins comprising a first and a second Fv for binding to a first and a second target epitope, wherein each Fv comprises a VL and a VH, and the VL comprises three CDRs flanked by framework sequences, and the VH comprises three CDRs flanked by framework sequences, wherein the first Fv specifically binds, under physiological conditions, to an epitope located on the extracellular part of a CD3-chain, and wherein the second Fv specifically binds, under physiological conditions, to an epitope located on target cells. The target cells can by any malignant cells such as cancer cells.

Each VL and VH herein comprises three complementarity determining regions (CDRs) flanked by framework sequences. Framework sequences are structurally conserved regions that normally tend to form a β-sheet structure positioning the CDRs for specific binding to the target epitope under physiological conditions.

The CDR sequences herein have been determined using the well-known Kabat system.

The first Framework sequence is N-terminal to the CDR1, the second Framework sequence is located between CDR1 and CDR2, while the third Framework sequence is located between CDR2 and CDR3. Accordingly, both a VL and VH can be roughly visualized as follows, with the CDRs boxed and the N-terminal indicated as N-:
N-FRAMEWORK1 [symbol] FRAMEWORK2 [symbol] FRAMEWORK3 [symbol] FRAMEWORK4

In the bispecific proteins herein, each of the framework sequences in the first Fv may independently be human, or murine or of any other suitable source.

A framework sequence is human if it is present in a human antibody, and it is flanking a CDR in a human VL or human VH, or it is encoded by a human genomic sequence flanking a CDR in a human VL-sequence or human VH-sequence.

Antibodies obtained from humans will have human framework sequences. However, it is well known that murine framework sequences can be completely or partially replaced by human framework sequences. This process is called humanization, and the resulting Fv is thus humanized.

In one embodiment, the human framework sequences are mature human framework sequences available from known human antibodies. Without being bound by theory, such framework sequences may convey very low risk of triggering unwanted immunogenic responses against the Fv in humans, and at the same time increase the likelihood of obtaining stable binding units which are expressed well in cellular systems.

In one embodiment, the Fv comprises or consists of VL-linker-VH. In another embodiment, the Fv comprises or consists of VH-linker-VL.

Such antigen binding domains are often referred to as single chain Fv-fragments (scFv's). The linker between VL and VH in a scFv generally needs a certain length in order to allow the VH and VL to form a functional antigen binding domain. In one embodiment, the linker comprises 10 to 50 amino acid residues. In one embodiment, the linker comprises 15 to 25 glycine and/or serine residues.

Alternatively, the VH and VL may be connected by a disulphide bridges or alternative linkers. The VL and VH may be embedded in a Fab-fragment of an antibody or an antibody as such.

Bispecific proteins for binding and recruiting T cells to target cells can have a variety of formats. Examples of such T cell-engagers are BiTEs. For example, Blinatumomab is a bispecific CD19-directed CD3 T-cell engager that binds to CD19 expressed on the surface of cells of B-lineage origin and CD3 expressed on the surface of T cells. It activates endogenous T cells by connecting CD3 in the T-cell receptor (TCR) complex with CD19 on benign and malignant B cells. Blinatumomab mediates the formation of a synapse between the T-cell and the tumor cell, upregulation of cell adhesion molecules, production of cytolytic proteins, release of inflammatory cytokines, and proliferation of T cells, which result in redirected lysis of CD19+ cells.

Three suitable BiTEs are disclosed by protein sequences SEQ ID NO: 2 (CD19 BiTE), SEQ ID NO: 3 (TP3 BiTE) and SEQ ID NO: 4 (CD22 BITE).

TP3 is a known murine antibody, and the TP3 Fv including TP3 scFv's are described in WO2020127734 (A1). These Fv's specifically bind, under physiological conditions, to an epitope located on osteosarcoma cells. Accordingly, such BiTEs may be used in treatment of solid tumors like osteosarcoma.

Many types of bispecific T-cell engagers are known. Some examples are described by Goebeler and Bargou (Nat. Rev. Clin. Oncol. 17(7):418-434 (2020); doi: 10.1038/s41571-020-0347-5), or by Zhou et al. (Biomarker Research 9(38) (2021).

Nucleic acids encoding and expressing proteins are well known, for example DNA and RNA. The proteins mentioned herein can be encoded by recombinant nucleic acids. Recombinant nucleic acids are non-naturally occurring nucleic acids.

The recombinant nucleic acids may comprise promoters, enhancers and other well-known elements for providing expression of the bispecific proteins in NK cells.

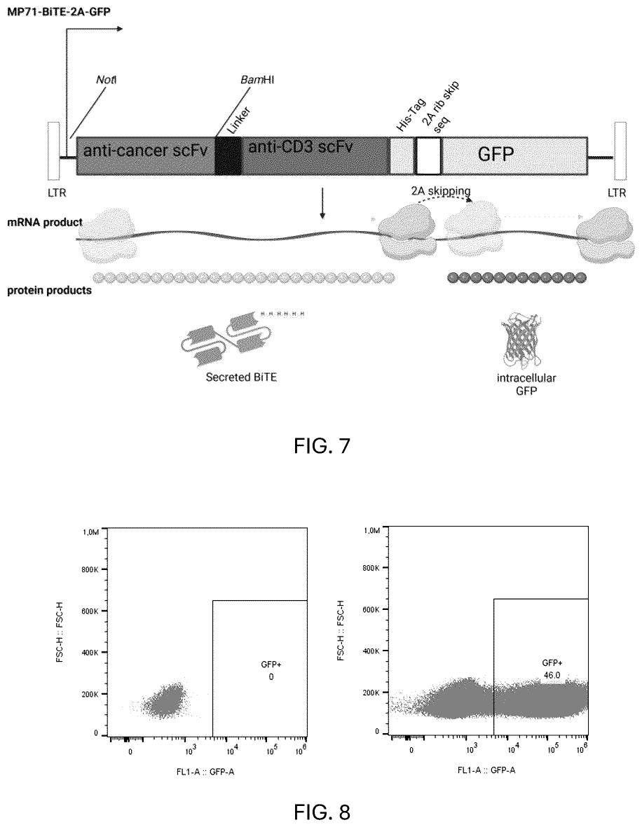

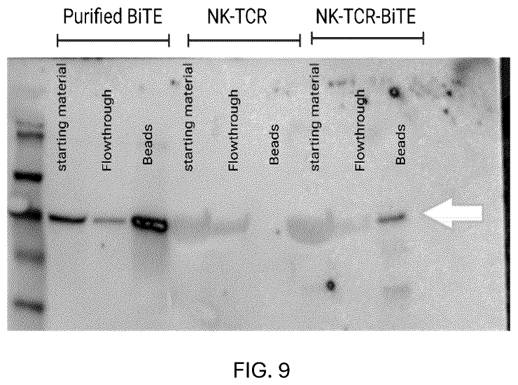

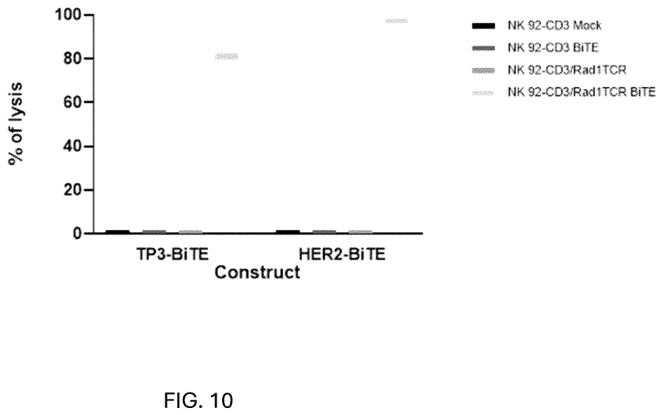

In order to achieve secretion of the bispecific proteins herein, the nucleic acids encoding them needs to contain a signal sequence (13-30 aa) that will target the BiTE protein to the secretory pathway and optionally a terminal His tag that can be used for either purification or detection (FIG. 7). The BiTE sequence was also cloned as a 2A-fusion with GFP in order to evaluate the ratio of BiTE producing NK-TCR in a given sample. The BiTE is normally transferred by retroviral vector in order to obtain a permanent production/secretion. The pMP71 system has been used for years and is acknowledged as efficient in lymphoid cells (Engels et al., Human Gene Therapy, 2003 August 10; 14(12, p. 1155-68). The transgene is transferred using the spinolculation method, and transduction efficiency is monitored by GFP detection. Secretion of the BiTE validation can be performed either directly by purifying the supernatant and running a Western blot from the enriched fraction (Ni-column, FIG. 9) or by killing target cells (i) expressing the BiTE specific antigen and (ii) not sensitive to NK cell killing. For example, BL41 are CD19+ and not sensitive to NK-92 cell killing.

Pharmaceutical compositions can comprise the KK cells and a pharmaceutically acceptable excipient. The pharmaceutical compositions herein can be a composition suitable for administration of therapeutic cells to a patient. The most common administration route for therapeutic cells is intravenous administration. Accordingly, said pharmaceutical compositions may for example be sterile aqueous solutions with a neutral pH. Said pharmaceutical compositions can contain pharmaceutically acceptable excipients suitable for their intended use. Accordingly, said pharmaceutical compositions may for example be sterile aqueous solutions with a physiological pH. The sterile pharmaceutical compositions may be a cell suspension for infusion comprising from 1×10⁶ NK cells to 1×10¹⁰ NK cells, such as 1×10⁷ NK cells to 1×10⁹ NK cells.

Such drugs can be for example be supplied in an infusion bag containing approximately 30 to 100 mL of a frozen suspension of the NK cells in 5% DMSO and 2.5% human serum albumin.

It is to be noted that the term “a” or “an” entity refers to one or more of that entity; for example, “a cell”, is understood to represent one or more cells. As such, the terms “a” (or “an”), “one or more”, and “at least one” can be used interchangeably herein. As any skilled person will understand, an NK-cell expressing a TCR-complex will normally express numerous such TCR-complexes in its cell membrane. Furthermore, an NK-cell expressing and secreting BiTEs will normally express and secrete numerous such BiTEs.

Physiological conditions, as used herein, means the environment encountered or simulated, in a living patient, where the disclosed cells and bispecific proteins are intended to bind an epitope located on target cells. In general, the physiological conditions in most human extracellular fluids are normally ca. 37° C., pH in the range of 6.0 to 7.5. However, in solid tumors, the physiological conditions i.e. the tumor microenvironment (TME) is often hypoxic and acidic.

The BiTE disclosed in the example comprise a well-known C-terminal oligopeptide comprising multiple histidine residues, also known as a “His-tag”, for purification and detection during research and early development. Such “His-tag” will not necessarily be present in a protein drug.

For promoting an understanding of the principles of the present disclosure, reference will now be made to embodiments illustrated herein and specific language will be used to describe the same. It will nevertheless be understood that no limitation of the scope is thereby intended. Any alterations and further modification in the described NK cells, the described methods for producing/preparing the NK cells, the described therapeutic compositions containing the NK cells, and the described therapeutics use of the NK cells and any further application of the principles of the invention as described herein, are contemplated as would normally occur to one skilled in the art within the present field.

As used herein, NK-TCR means NK cells expressing a TCR-complex.

As used herein, NK-TCR-BiTE means NK cells expressing a TCR-complex and secreting a BiTE.

As used herein, target cells are malignant or otherwise undesired cells, e.g. cancer cells or autoimmune cells, which do not express TCR-complexes in their cell membrane in any significant amount.

### Sequences

BiTE anti-CD19:

DNA sequence of the full length MP71-CD19-BiTE
retroviral vector (SEQ ID NO: 1):
GGCCGCCCCCTTCACCATGGAGACCGACACCCTGCTGCTGTGGGTGCTGC
TGCTGTGGGTGCCAGGCAGCACCGGCGACATCCAGATGACCCAGACCACC
AGCAGCCTGAGCGCCAGCCTGGGCGACCGGGTGACCATCAGCTGCAGAGC
CAGCCAGGACATCAGCAAGTACCTGAACTGGTACCAGCAGAAGCCCGACG
GCACCGTGAAGCTGCTGATCTACCACACCAGCCGGCTGCACAGCGGCGTG
CCCAGCCGGTTCAGCGGCAGCGGCAGCGGCACCGACTACAGCCTGACCAT
CAGCAACCTGGAGCAGGAGGACATCGCCACCTACTTCTGCCAGCAGGGCA
ACACCCTGCCCTACACCTTCGGAGGCGGCACCAAGCTGGAGATCACCAAG
GCCGGAGGCGGAGGCTCTGGCGGAGGCGGCTCTGGCGGAGGCGGCTCTGG
CGGAGGCGGCAGCGAGGTGAAGCTGCAGGAGTCTGGCCCAGGCCTGGTGG
CCCCAAGCCAGAGCCTGAGCGTGACCTGCACCGTGAGCGGCGTGAGCCTG
CCCGACTACGGCGTGAGCTGGATCAGGCAGCCCCCACGGAAGGGCCTGGA
GTGGCTGGGCGTGATCTGGGGCAGCGAGACCACCTACTACAACAGCGCCC
TGAAGAGCCGGCTGACCATCATCAAGGACAACAGCAAGAGCCAGGTGTTC
CTGAAGATGAACAGCCTGCAGACCGACGACACCGCCATCTACTACTGCGC
CAAGCACTACTACTATGGCGGCAGCTACGCTATGGACTACTGGGGCCAGG
GCACCAGCGTGACCGTGAGCTCGGATCCCGGTGGGGGAGGATCAGACATC
AAGCTGCAACAATCTGGCGCTGAGTTGGCGAGGCCGGGGGCGAGTGTTAA
GATGAGTTGTAAGACTAGTGGTTATACTTTCACCAGGTACACCATGCACT
GGGTGAAGCAGCGCCCGGGACAGGGTCTTGAATGGATAGGTTATATCAAT
CCGTCCCGAGGGTATACTAACTACAACCAAAAATTCAAAGACAAAGCGAC
TCTGACTACGGACAAATCCTCTAGCACAGCATACATGCAGCTTTCCAGCC
TCACTAGTGAAGACTCAGCAGTTTACTATTGTGCTAGGTACTATGATGAT
CACTATTGCCTCGATTACTGGGGACAAGGGACGACCCTCACGGTGTCAAG
TGTGGAGGGTGGCTCCGGAGGTTCCGGAGGTTCCGGCGGGAGCGGTGGAG
TGGATGATATCCAACTTACTCAATCCCCGGCCATAATGAGTGCAAGTCCA
GGAGAAAAGGTGACAATGACTTGCCGAGCGTCATCATCCGTATCTTACAT
GAACTGGTACCAGCAGAAGAGTGGCACGTCTCCTAAGCGATGGATATACG
ACACATCCAAGGTCGCAAGCGGAGTGCCGTACCGATTCAGCGGTTCTGGG
TCTGGGACTAGTTACTCCTTGACTATCTCCTCAATGGAAGCCGAAGACGC
CGCGACATACTATTGTCAACAATGGTCCTCCAACCCCCTTACGTTCGGTG
CTGGAACAAAGTTGGAGCTGAAACATCACCACCATCACCACAGAGCCAAG
AGAGGCAGCGGCGCCACCAACTTCAGCCTGCTGAAGCAGGCCGGCGACGT
GGAAGAGAACCCTGGACCAATGGTGAGCAAGGGCGAGGAGCTGTTCACCG
GGGTGGTGCCCATCCTGGTCGAGCTGGACGGCGACGTAAACGGCCACAAG
TTCAGCGTGTCCGGCGAGGGCGAGGGCGATGCCACCTACGGCAAGCTGAC
CCTGAAGTTCATCTGCACCACCGGCAAGCTGCCCGTGCCCTGGCCCACCC
TCGTGACCACCCTGACCTACGGCGTGCAGTGCTTCAGCCGCTACCCCGAC
CACATGAAGCAGCACGACTTCTTCAAGTCCGCCATGCCCGAAGGCTACGT
CCAGGAGCGCACCATCTTCTTCAAGGACGACGGCAACTACAAGACCCGCG
CCGAGGTGAAGTTCGAGGGCGACACCCTGGTGAACCGCATCGAGCTGAAG
GGCATCGACTTCAAGGAGGACGGCAACATCCTGGGGCACAAGCTGGAGTA
CAACTACAACAGCCACAACGTCTATATCATGGCCGACAAGCAGAAGAACG
GCATCAAGGTGAACTTCAAGATCCGCCACAACATCGAGGACGGCAGCGTG
CAGCTCGCCGACCACTACCAGCAGAACACCCCCATCGGCGACGGCCCCGT
GCTGCTGCCCGACAACCACTACCTGAGCACCCAGTCCGCCCTGAGCAAAG
ACCCCAACGAGAAGCGCGATCACATGGTCCTGCTGGAGTTCGTGACCGCC
GCCGGGATCACTCTCGGCATGGACGAGCTGTACAAGTAATGACTCGAGAA
GGGTGGGCGCGCCGACCCAGCTTTCTTGTACAAAGTGGTTGATCTAGAGG
GCCCGCGGTTCGAAGGTAAGCCTATCCCTAACCCTCAATTCGAGCATCTT
ACCGCCATTTATTCCCATATTTGTTCTGTTTTTCTTGATTTGGGTATACA
TTTAAATGTTAATAAAACAAAATGGTGGGGCAATCATTTACATTTTATGG
GATATGTAATTACTAGTTCAGGTGTATTGCCACAAGACAAACATGTTAAG
AAACTTTCCCGTTATTTACGCTCTGTTCCTGTTAATCAACCTCTGGATTA
CAAAATTTGTGAAAGATTGACTGATATTCTTAACTATGTTGCTCCTTTTA
CGCTGTGTGGATATGCTGCTTTAATGCCTCTGTATCATGCTATTGCTTCC
CGTACGGCTTTCGTTTTCTCCTCCTTGTATAAATCCTGGTTGCTGTCTCT
TTATGAGGAGTTGTGGCCCGTTGTCCGTCAACGTGGCGTGGTGTGCTCTG
TGTTTGCTGACGCAACCCCCACTGGCTGGGGCATTGCCACCACCTGTCAA
CTCCTTTCTGGGACTTTCGCTTTCCCCCTCCCGATCGCCACGGCAGAACT
CATCGCCGCCTGCCTTGCCCGCTGCTGGACAGGGGCTAGGTTGCTGGGCA
CTGATAATTCCGTGGTGTTGTCGGGGAAGCTGACGTCCTTTCCATGGCTG
CTCGCCTGTGTTGCCAACTGGATCCTGCGCGGGACGTCCTTCTGCTACGT
CCCTTCGGCTCTCAATCCAGCGGACCTCCCTTCCCGAGGCCTTCTGCCGG
TTCTGCGGCCTCTCCCGCGTCTTCGCTTTCGGCCTCCGACGAGTCGGATC
TCCCTTTGGGCCGCCTCCCCGCCTGTTTCGCCTCGGCGTCCGGTCCGTGT
TGCTTGGTCGTCACCTGTGCAGAATTGCGAACCATGGATTCCACCGTGAA
CTTTGTCTCCTGGCATGCAAATCGTCAACTTGGCATGCCAAGAATTAATT
CGGATCCAAGCTTAGGCCTGCTCGCTTTCTTGCTGTCCCATTTCTATTAA
AGGTTCCTTTGTTCCCTAAGTCCAACTACTAAACTGGGGGATATTATGAA
GGGCCTTGAGCATCTGGATTCTGCCTAGCGCTAAGCTTAACACGAGCCAT
AGATAGAATAAAAGATTTTATTTAGTCTCCAGAAAAAGGGGGGAATGAAA
GACCCCACCTGTAGGTTTGGCAAGCTAGCTTAAGTAACGCCATTTTGCAA
GGCATGGAAAATACATAACTGAGAATAGAGAAGTTCAGATCAAGGTTAGG
AACAGAGAGACAGCAGAATATGGGCCAAACAGGATATCTGTGGTAAGCAG
TTCCTGCCCCGGCTCAGGGCCAAGAACAGTTGGAACAGCAGAATATGGGC
CAAACAGGATATCTGTGGTAAGCAGTTCCTGCCCCGGCTCAGGGCCAAGA
ACAGATGGTCCCCAGATGCGGTCCCGCCCTCAGCAGTTTCTAGAGAACCA
TCAGATGTTTCCAGGGTGCCCCAAGGACCTGAAATGACCCTGTGCCTTAT
TTGAACTAACCAATCAGTTCGCTTCTCGCTTCTGTTCGCGCGCTTCTGCT
CCCCGAGCTCAATAAAAGAGCCCACAACCCCTCACTCGGCGCGCCAGTCC
TCCGATAGACTGCGTCGCCCGGGTACCCGTGTTCTCAATAAACCCTCTTG
CAGTTGCATCCGACTCGTGGTCTCGCTGTTCCTTGGGAGGGTCTCCTCTG
AGTGATTGACTGCCCACCTCGGGGGTCTTTCATTCTCGAGCAGCTTGGCG
TAATCATGGTCATAGCTGTTTCCTGTGTGAAATTGTTATCCGCTCACAAT
TCCACACAACATACGAGCCGGAAGCATAAAGTGTAAAGCCTGGGGTGCCT
AATGAGTGAGCTAACTCACATTAATTGCGTTGCGCTCACTGCCCGCTTTC
CAGTCGGGAAACCTGTCGTGCCAGCTGCATTAATGAATCGGCCAACGCGC
GGGGAGAGGCGGTTTGCGTATTGGGCGCTCTTCCGCTTCCTCGCTCACTG
ACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAAT
CGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGC
CGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTC
CAAGCTGGGCTGTGTGCACGAACCCCCCGTTCAGCCCGACCGCTGCGCCT
TATCCGGTAACTATCGTCTTGAGTCCAACCCGGTAAGACACGACTTATCG
CCACTGGCAGCAGCCACTGGTAACAGGATTAGCAGAGCGAGGTATGTAGG
CGGTGCTACAGAGTTCTTGAAGTGGTGGCCTAACTACGGCTACACTAGAA
GAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAAC
TCACGTTAAGGGATTTTGGTCATGAGATTATCAAAAAGGATCTTCACCTA
GATCCTTTTAAATTAAAAATGAAGTTTTAAATCAATCTAAAGTATATATG
AGTAAACTTGGTCTGACAGTTACCAATGCTTAATCAGTGAGGCACCTATC
TCAGCGATCTGTCTATTTCGTTCATCCATAGTTGCCTGACTCCCCGTCGT
GTAGATAACTACGATACGGGAGGGCTTACCATCTGGCCCCAGTGCTGCAA
TGATACCGCGAGACCCACGCTCACCGGCTCCAGATTTATCAGCAATAAAC
CAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGC
CAGTTAATAGTTTGCGCAACGTTGTTGCCATTGCTACAGGCATCGTGGTG
TCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCT
TCGGTCCTCCGATCGTTGTCAGAAGTAAGTTGGCCGCAGTGTTATCACTC
ATGGTTATGGCAGCACTGCATAATTCTCTTACTGTCATGCCATCCGTAAG
ATGCTTTTCTGTGACTGGTGAGTACTCAACCAAGTCATTCTGAGAATAGT
GTATGCGGCGACCGAGTTGCTCTTGCCCGGCGTCAATACGGGATAATACC
GCGCCACATAGCAGAACTTTAAAAGTGCTCATCATTGGAAAACGTTCTTC
GGGGCGAAAACTCTCAAGGATCTTACCGCTGTTGAGATCCAGTTCGATGT
AACCCACTCGTGCACCCAACTGATCTTCAGCATCTTTTACTTTCACCAGC
GTTTCTGGGTGAGCAAAAACAGGAAGGCAAAATGCCGCAAAAAAGGGAAT
AAGGGCGACACGGAAATGTTGAATACTCATACTCTTCCTTTTTCAATATT
ATTGAAGCATTTATCAGGGTTATTGTCTCATGAGCGGATACATATTTGAA
TGTATTTAGAAAAATAAACAAATAGGGGTTCCGCGCACATTTCCCCGAAA
AGTGCCACCTGACGTCTAAGAAACCATTATTATCATGACATTAACCTATA
AAAATAGGCGTATCACGAGGCCCTTTCGTCTCGCGCGTTTCGGTGATGAC
GGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTG
TTGGCGGGTGTCGGGGCTGGCTTAACTATGCGGCATCAGAGCAGATTGTA
CTGAGAGTGCACCATATGCGGTGTGAAATACCGCACAGATGCGTAAGGAG
AAAATACCGCATCAGGCGCCATTCGCCATTCAGGCTGCGCAACTGTTGGG
AAGGGCGATCGGTGCGGGCCTCTTCGCTATTACGCCAGCTGGCGAAAGGG
GGATGTGCTGCAAGGCGATTAAGTTGGGTAACGCCAGGGTTTTCCCAGTC
ACGACGTTGTAAAACGACGGCCAGTGAATTAGTACTCTAGCTTAAGTAAC
GCCATTTTGCAAGGCATGGAAAATACATAACTGAGAATAGAGAAGTTCAG
ATCAAGGTTAGGAACAGAGAGACAGCAGAATATGGGCCAAACAGGATATC
TGTGGTAAGCAGTTCCTGCCCCGGCTCAGGGCCAAGAACAGTTGGAACAG
CAGAATATGGGCCAAACAGGATATCTGTGGTAAGCAGTTCCTGCCCCGGC
TCAGGGCCAAGAACAGATGGTCCCCAGATGCGGTCCCGCCCTCAGCAGTT
TCTAGAGAACCATCAGATGTTTCCAGGGTGCCCCAAGGACCTGAAATGAC
CCTGTGCCTTATTTGAACTAACCAATCAGTTCGCTTCTCGCTTCTGTTCG
CGCGCTTCTGCTCCCCGAGCTCAATAAAAGAGCCCACAACCCCTCACTCG
GCGCGCCAGTCCTCCGATAGACTGCGTCGCCCGGGTACCCGTATTCCCAA
TAAAGCCTCTTGCTGTTTGCATCCGAATCGTGGACTCGCTGATCCTTGGG
AGGGTCTCCTCAGATTGATTGACTGCCCACCTCGGGGGTCTTTCATTTGG
AGGTTCCACCGAGATTTGGAGACCCCTGCCCAGGGACCACCGACCCCCCC
GCCGGGAGGTAAGCTGGCCAGCGGTCGTTTCGTGTCTGTCTCTGTCTTTG
TGCGTGTTTGTGCCGGCATCTAATGTTTGCGCCTGCGTCTGTACTAGTTG
GCTAACTAGATCTGTATCTGGCGGTCCCGCGGAAGAACTGACGAGTTCGT
ATTCCCGGCCGCAGCCCCTGGGAGACGTCCCAGCGGCCTCGGGGGCCCGT
TTTGTGGCCCATTCTGTATCAGTTAACCTACCCGAGTCGGACTTTTTGGA
GCTCCGCCACTGTCCGAGGGGTACGTGGCTTTGTTGGGGGACGAGAGACA
GAGACACTTCCCGCCCCCGTCTGAATTTTTGCTTTCGGTTTTACGCCGAA
ACCGCGCCGCGCGTCTTGTCTGCTGCAGCATCGTTCTGTGTTGTCTCTGT
CTGACTGTGTTTCTGTATTTGTCTGAAAATTAGCTCGACAAAGTTAAGTA
ATAGTCCCTCTCTCCAAGCTCACTTACAGGCGGCCTCGAGTAATACGACT
CACTATAGGGAGACCCAAGCTGGCTAGGTAAGCTTGATCAACAAGTTTGT
ACAAAAAAGCAGGCTCCGC
protein sequence of CD19-BiTE (SEQ ID NO: 2)
METDTLLLWVLLLWVPGSTGDIQMTQTTSSLSASLGDRVTISCRASQDIS
KYLNWYQQKPDGTVKLLIYHTSRLHSGVPSRFSGSGSGTDYSLTISNLEQ
EDIATYFCQQGNTLPYTFGGGTKLEITKAGGGGSGGGGSGGGGSGGGGSE
VKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYY
GGSYAMDYWGQGTSVTVSSDPGGGGSDIKLQQSGAELARPGASVKMSCKT
SGYTFTRYTMHWVKQRPGQGLEWIGYINPSRGYTNYNQKFKDKATLTTDK
SSSTAYMQLSSLTSEDSAVYYCARYYDDHYCLDYWGQGTTLTVSSVEGGS
GGSGGSGGSGGVDDIQLTQSPAIMSASPGEKVTMTCRASSSVSYMNWYQQ
KSGTSPKRWIYDTSKVASGVPYRFSGSGSGTSYSLTISSMEAEDAATYYC
QQWSSNPLTFGAGTKLELKHHHHHH
protein sequence of TP3 BiTE (SEQ ID NO: 3)
METDTLLLWVLLLWVPGSTGDIVLTQSPASLAVSLGQRATISCRASKSVS
TGYSYLHWYQQKPGQPPKLLIYLASNLESGVPARFSGSGSGTDFTLNIHP
VEEEDAATYYCQHSRELPLTFGAGTKLELKRGGGGSGGGGSGGGGSGGGG
SEVQLQQSGAELVKPGASVKISCKASGYTFTDYNMDWVKQSHGKSLEWIG
DINPNYDSTRYNQKFKGKATLTVDKSSSTAYMELRSLTSEDTAVYYCARG
DYYVSSYGHDYAMDYWGQGTSVTVSSDPGGGGSDIKLQQSGAELARPGAS
VKMSCKTSGYTFTRYTMHWVKQRPGQGLEWIGYINPSRGYTNYNQKFKDK
ATLTTDKSSSTAYMQLSSLTSEDSAVYYCARYYDDHYCLDYWGQGTTLTV
SSVEGGSGGSGGSGGSGGVDDIQLTQSPAIMSASPGEKVTMTCRASSSVS
YMNWYQQKSGTSPKRWIYDTSKVASGVPYRFSGSGSGTSYSLTISSMEAE
DAATYYCQQWSSNPLTFGAGTKLELKHHHHHH
protein sequence of CD22 BiTE (clone RFB-4)
(SEQ ID NO: 4)
METDTLLLWVLLLWVPGSTGEVQLVESGGGLVKPGGSLKLSCAASGFAFS
IYDMSWVRQTPEKCLEWVAYISSGGGTTYYPDTVKGRFTISRDNAKNTLY
LQMSSLKSEDTAMYYCARHSGYGTHWGVLFAYWGQGTLVTVSAGGGGSGG
GGSGGGGSDIQMTQTTSSLSASLGDRVTISCRASQDISNYLNWYQQKPDG
TVKLLIYYTSILHSGVPSRFSGSGSGTDYSLTISNLEQEDFATYFCQQGN
TLPWTFGCGTKLEIKSDPGGGGSDIKLQQSGAELARPGASVKMSCKTSGY
TFTRYTMHWVKQRPGQGLEWIGYINPSRGYTNYNQKFKDKATLTTDKSSS
TAYMQLSSLTSEDSAVYYCARYYDDHYCLDYWGQGTTLTVSSVEGGSGGS
GGSGGSGGVDDIQLTQSPAIMSASPGEKVTMTCRASSSVSYMNWYQQKSG
TSPKRWIYDTSKVASGVPYRFSGSGSGTSYSLTISSMEAEDAATYYCQQW
SSNPLTFGAGTKLELKHHHHHH
protein sequence of the HER2 scFv (SEQ ID NO: 5)
METDTLLLWVLLLWVPGSTGDIQMTQSPSSLSASVGDRVTITCRASQDVN
TAVAWYQQKPGKAPKLLIYSASFLYSGVPSRFSGSRSGTDFTLTISSLQP
EDFATYYCQQHYTTPPTFGQGTKVEIKGSTSGGGSGGGSGGGGSSEVQLV
ESGGGLVQPGGSLRLSCAASGFNIKDTYIHWVRQAPGKGLEWVARIYPTN
GYTRYADSVKGRFTISADTSKNTAYLQMNSLRAEDTAVYYCSRWGGDGFY
AMDYWGQGTLVTVSS

underline: signal sequence
italic: scFv intra- and inter-linkers
Bold: His tag

### EXAMPLES

### Example 1. Production of NK-TCR CD19 BITE

A retrovirus containing a transgene encoding for the anti-CD19 BiTE 2A-linked with GFP was produced into Hek cells. The supernatant of the Hek cells was harvested twice and stocked at 4° C. The supernatant was used to transduce NK-TCR cells. These NK-TCR were produced as previously depicted (Mensali et al. 2019) using a polycistronic CD3 construct and a human TCR (Radium-1, Inderberg et al. 2017). The transduction efficiency was monitored using GFP as a transduction marker.

### Example 2 Expression of BiTE

NK-TCR cells transduced or not with CD19 BiTE were run into a flowcytometer and fluorescence was detected using laser excitement at 488 nm and detected at 510 nm. Expression of BiTE in NK cells by GFP detection is shown in FIG. 8.

Expression of BiTE in NK cells by purification in the culture medium. NK-TCR-BiTE and NK-TCR were grown in NK medium at 1M/mL for 2 days and 1 mL of supernatant was harvested. Nickel slurry was used to pull down His-tagged BiTE from the supernatant and each fraction was separated by SDS-PAGE and transferred to a nitrocellulose membrane for Western blot detection using an anti-His antibody. As a control, a validated BiTE solution was used. For each test, the first fraction is the starting material which is not detected for the NK supernatants due to a concentration below detection level. The flowthrough of the slurry represents what is not bound, and here again, NK supernatants are negative whereas the large-scale beads seems to be more concentrated than the slurry's capacity, hence the presence of BiTE in this fraction. Finally, the beads were also run, they should contain the attached fraction, concentrated on the beads. As shown in FIG. 9, only the positive control and the NK-TCR-BiTE beads contain BiTEs. Thus NK-TCR-BiTE release BiTEs into their supernatant.

### Example 3 BiTE Specificity and Killing

In order to validate that the released BiTE is able to redirect CD3 positive effector cells, we have mixed NK-TCR-BiTE or NK-TCR with B lymphoma cell line BL41, which is CD19 positive. As shown and previously published (Walseng et al 2019), NK-92 are not able to kill BL-41, however, when anti-CD19 BiTE is secreted, NK-TCR where able to efficiently kill CD19-positive targets. This teaches us that (i) BiTEs released by NK cells are functional and (ii) NK cells expressing a TCR-CD3 complex can be redirected by BiTE.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A Natural Killer (NK)-cell comprising nucleic acids for expressing an αβ T-cell Receptor (TCR), CD3ζ, CD3γ, CD3δ and CD3ε in its cell membrane, and a nucleic acid for expressing and secreting a bispecific protein,
wherein the bispecific protein comprises a first Fv for binding to a first target epitope and a second Fv for binding to a second target epitope,
wherein the first Fv specifically binds, under physiological conditions, to an epitope located on the extracellular part of a CD3-chain, and
wherein the second Fv specifically binds, under physiological conditions, to an epitope located on target cells.

2. The NK-cell according to claim 1, wherein the TCR and the bispecific protein recognize different epitopes of the same target protein from the same target cells.

3. The NK-cell according to claim 1, wherein the TCR and the bispecific protein recognize epitopes of different target proteins from the same target cells.

4. The NK-cell according to claim 1, wherein the TCR specifically binds, under physiological conditions, to a peptide from Melanoma-associated antigen 4 presented on an HLA,
and
the second Fv specifically binds, under physiological conditions, to an extracellular target epitope located on a human cancer cell selected from a group consisting of Non-Small Cell Lung Cancer, Ovarian cancer, melanoma cancer, synovial sarcoma and gastroesophageal cancers.

5. The NK-cell according to claim 1, wherein the TCR specifically binds, under physiological conditions, to a peptide from Kita-Kyushu Lung Cancer Antigen-1 presented on an HLA,
and
the second Fv specifically binds, under physiological conditions, to an extracellular target epitope located on a human cancer cell selected from a group consisting of breast cancer, gastric cancer, lung cancer, pancreatic cancer and cervix cancer.

6. The NK-cell according to claim 1, wherein the TCR specifically binds, under physiological conditions, to a peptide from PRAME presented on an HLA,
and
the second Fv specifically binds, under physiological conditions, to an extracellular target epitope located on a human melanoma cell.

7. The NK-cell according to claim 1, wherein the first Fv specifically binds, under physiological conditions, to an extracellular epitope located on a human CD3ε protein.

8. The NK-cell according to claim 1, wherein the second Fv specifically binds, under physiological conditions, to an extracellular epitope located on human cancer cells of solid tumor origin.

9. The NK-cell according to claim 1, wherein the first and/or second Fv is a single-chain Fv (scFv).

10. The NK-cell according to claim 1, wherein the NK-cell is a human cell line or human primary cell.

11. A cell population comprising the cells according to claim 1.

12. An aqueous pharmaceutical composition for intravenous or intratumoral administration comprising the cells according to claim 1 or the cell population according to claim 11, wherein the composition is sterile and/or isotonic.

13. A method of treatment of solid tumors comprising the step of administering the pharmaceutical composition according to claim 12 to a human patient in need thereof.


SEQUENCE LISTING (5 sequences)

SEQ ID NO: 1 - DNA, 8019 bp, synthetic construct
  source 1..8019: mol_type = other DNA
ggccgcccccttcaccatggagaccgacaccctgctgctgtgggtgctgctgctgtgggtgccaggcagcaccggcgacatccagatgacccagaccaccagcagcctgagcgccagcctgggcgaccgggtgaccatcagctgcagagccagccaggacatcagcaagtacctgaactggtaccagcagaagcccgacggcaccgtgaagctgctgatctaccacaccagccggctgcacagcggcgtgcccagccggttcagcggcagcggcagcggcaccgactacagcctgaccatcagcaacctggagcaggaggacatcgccacctacttctgccagcagggcaacaccctgccctacaccttcggaggcggcaccaagctggagatcaccaaggccggaggcggaggctctggcggaggcggctctggcggaggcggctctggcggaggcggcagcgaggtgaagctgcaggagtctggcccaggcctggtggccccaagccagagcctgagcgtgacctgcaccgtgagcggcgtgagcctgcccgactacggcgtgagctggatcaggcagcccccacggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactatggcggcagctacgctatggactactggggccagggcaccagcgtgaccgtgagctcggatcccggtgggggaggatcagacatcaagctgcaacaatctggcgctgagttggcgaggccgggggcgagtgttaagatgagttgtaagactagtggttatactttcaccaggtacaccatgcactgggtgaagcagcgcccgggacagggtcttgaatggataggttatatcaatccgtcccgagggtatactaactacaaccaaaaattcaaagacaaagcgactctgactacggacaaatcctctagcacagcatacatgcagctttccagcctcactagtgaagactcagcagtttactattgtgctaggtactatgatgatcactattgcctcgattactggggacaagggacgaccctcacggtgtcaagtgtggagggtggctccggaggttccggaggttccggcgggagcggtggagtggatgatatccaacttactcaatccccggccataatgagtgcaagtccaggagaaaaggtgacaatgacttgccgagcgtcatcatccgtatcttacatgaactggtaccagcagaagagtggcacgtctcctaagcgatggatatacgacacatccaaggtcgcaagcggagtgccgtaccgattcagcggttctgggtctgggactagttactccttgactatctcctcaatggaagccgaagacgccgcgacatactattgtcaacaatggtcctccaacccccttacgttcggtgctggaacaaagttggagctgaaacatcaccaccatcaccacagagccaagagaggcagcggcgccaccaacttcagcctgctgaagcaggccggcgacgtggaagagaaccctggaccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgactcgagaagggtgggcgcgccgacccagctttcttgtacaaagtggttgatctagagggcccgcggttcgaaggtaagcctatccctaaccctcaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggcctcgagtaatacgactcactatagggagacccaagctggctaggtaagcttgatcaacaagtttgtacaaaaaagcaggctccgc

SEQ ID NO: 2 - protein, 525 aa, synthetic construct
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 3 - protein, 532 aa, synthetic construct
METDTLLLWVLLLWVPGSTGDIVLTQSPASLAVSLGQRATISCRASKSVSTGYSYLHWYQQKPGQPPKLLIYLASNLESGVPARFSGSGSGTDFTLNIHPVEEEDAATYYCQHSRELPLTFGAGTKLELKRGGGGSGGGGSGGGGSGGGGSEVQLQQSGAELVKPGASVKISCKASGYTFTDYNMDWVKQSHGKSLEWIGDINPNYDSTRYNQKFKGKATLTVDKSSSTAYMELRSLTSEDTAVYYCARGDYYVSSYGHDYAMDYWGQGTSVTVSSDPGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 4 - protein, 522 aa, synthetic construct
METDTLLLWVLLLWVPGSTGEVQLVESGGGLVKPGGSLKLSCAASGFAFSIYDMSWVRQTPEKCLEWVAYISSGGGTTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGCGTKLEIKSDPGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 5 - protein, 265 aa, synthetic construct
METDTLLLWVLLLWVPGSTGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS